(19) United States
(12) Patent Application Publication
(10) Pub. No.: US 2017/0202786 A1
(43) Pub. Date: Jul. 20, 2017

(54) NOVEL PULSATILE DRUG DELIVERY SYSTEM

(71) Applicant: Jayendrakumar Dasharathlal Patel, Vijapur (IN)

(72) Inventors: Jayendrakumar Dasharathlal Patel, Vijapur (IN); Dasharathbhai Trikambhai Patel, Vijapur (IN); Shwetaben Dasharathbhai Patel, Vijapur (IN)

(73) Assignees: Pyrrhic Pharma Pvt Ltd, Vijapur (IN); Jayendrakumar Dasharathlal Patel, Vijapur (IN)

(21) Appl. No.: 15/002,388

(22) Filed: Jan. 20, 2016

(51) Int. Cl. A61K 31/135; A61K 9/20; A61K 9/28; A61K 9/00

(52) U.S. Cl. CPC A61K 31/135; A61K 9/0004; A61K 9/2018; A61K 9/2054; A61K 9/2013; A61K 9/2059; A61K 9/2866; A61K 9/2826; A61K 9/284; A61K 9/2086; A61K 9/2893; A61K 9/2095

(57) ABSTRACT

A novel pulsatile drug delivery system composition, methods of administration, method of treating disease condition and methods of making the same are provided. Novel pulsatile drug delivery systems provide adjustable lag time of release of drug from dosage form before administration. Novel pulsatile drug delivery systems provide administration of dosage form after modifying lag time of drug release while patient miss the administration of dosage form at particular time.

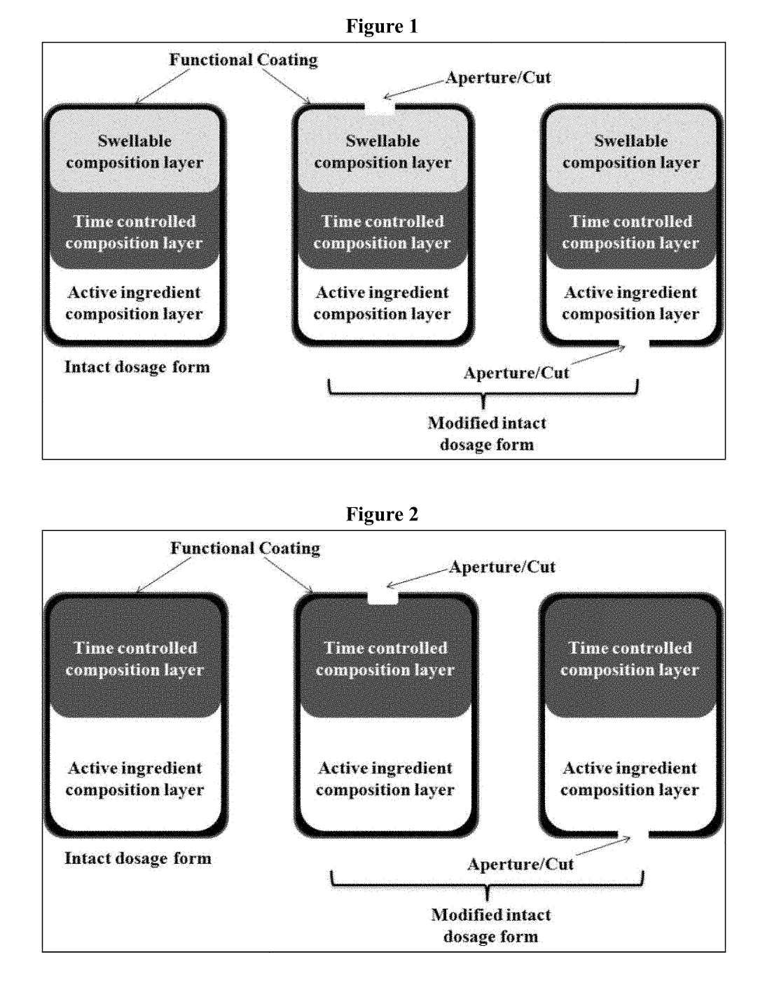

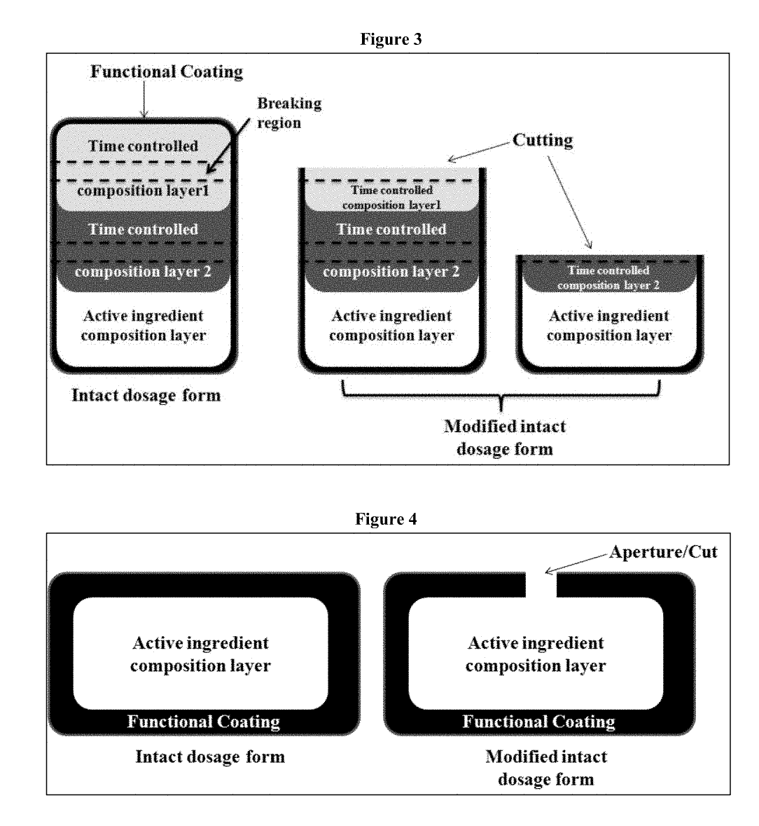

### CROSS REFERENCE TO RELATED APPLICATION

This application claims the benefits of Indian patent provisional application No. 2397/MUM/2014, filed on 25 Jul. 2014, which is incorporated herein by reference.

### FIELD OF THE INVENTION

A present invention is relates to a novel pulsatile drug delivery system composition, methods of administration, method of treating disease condition and methods of making a novel pulsatile drug delivery system having modifiable lag time for release of drug from dosage form before administration.

### BACKGROUND OF THE INVENTION

Oral dosage forms are known which provide a zero order or first order release in which the drug is released at a substantially steady rate per unit of time. These dosage forms are satisfactory for the administration of pharmaceutical dosage forms of many drugs. However, there are instances where maintaining a constant blood level of a drug is not desirable. In such cases (e.g., optimization of chemotherapy, reducing nocturnal or early morning systems of chronic diseases such as chrono pharmacological behavior where night time dosing is required, ischemic heart disease, asthma, arthritis, avoiding developing a tolerance to nitrates, antibiotics and steroidal contraceptives, drugs having high first-pass effect or where absorption windows exist), a ‘time controlled’ pulsatile drug delivery system may be more advantageous. There are also instances in which a ‘position-controlled’ drug delivery system (e.g. treatment of colon disease or use of colon as an absorption site for peptide and protein based products) may prove to be more efficacious.

In recent year, pulsatile systems are gaining a lot of interest as they deliver the drug at the right site of action at the right time and in the right amount, thus providing spatial and temporal delivery and increasing patient compliance. These systems are designed according to the circadian rhythm of the body. The release of the drug as a pulse after a lag time has to be designed in such a way that a complete and immediate or controlled drug release follows the lag time. Various systems like capsular systems, osmotic systems, single and multiple-unit systems based on the use of soluble or erodible polymer coating and use of rupturable membranes are developed.

U.S. Pat. No. 4,851,229 issued on Jul. 25, 1989 to Magruder et al. discusses relative merits of steady state versus pulsatile drug delivery regimens and discloses a unit dosage form of complex structure for pulsatile delivery of drug employing an osmotic pump mechanism and a semi permeable shell with a tiny hole in the shell through which the drug is ejected.

A pulsatile drug delivery system has been described in U.S. Pat. No. 5,472,708, which uses a minor portion of water-soluble polymer and a permeability reducing agent added to the water-insoluble coating to guarantee drug release. Drug delivery is controlled by the rupture of the membrane. The system provides a pulsatile input of drug into the intestine with the timing of release regulated by the thickness of coating and the amount of water-soluble polymer.

However, many of the formulations in the art rely on complex structures which can add to the cost of the manufacture of the drug and/or can be subject to malfunction leading to incorrect/inappropriate administration of the drug.

Pulsatile drug delivery systems found in literature teach about dosage from that release the drug after predefine lag time. In other word, lag time of drug release from dosage form cannot be adjusted before administration of dosage form. Pulsatile dosage form without modifiable lag time of drug release before administration is not useful while patient miss administration of dosage form at particular time period. For example, if patient need a drug at approximately 4 am on morning, then they would administer dosage form having 8 hours lag time at 8 pm on early night. But if patient miss the dosage form for administration at 8 pm on early night and remember at around 11 pm, in such case dosage form is not useful because it deliver the drug at around 7 am on morning. Present invention provide a novel pulsatile drug delivery system having modifiable lag time for release of drug or drug from dosage form before administration of dosage form, which is a advantage over previously founded pulsatile drug delivery system. Present invention also provide methods of administration of a novel pulsatile drug delivery system, method of treating disease condition with a novel pulsatile drug delivery system and methods of making a novel pulsatile drug delivery system having modifiable lag time for release of drug from dosage form before administration.

### OBJECTS OF THE INVENTION

It is an object of present invention to provide method of treating disease condition with pulsatile drug delivery system comprising administration of intact dosage form after modifying lag time of drug release; wherein said modified intact dosages form provide smaller lag time for release of drug from dosage form compared to intact dosage form having predefined lag time for release of drug.

It is an object of present invention to provide method of treating disease condition with pulsatile drug delivery system, wherein lag time for release of drug from pulsatile drug delivery system is modifiable before administration.

It is an object of present invention to provide method of treating disease condition with pulsatile drug delivery system having modifiable lag time of drug release, wherein a said pulsatile drug delivery system is useful when patient miss administration of dosage form at particular time period.

It is an object of present invention to design a novel pulsatile drug delivery system which is capable to providing one or more immediate release or controlled release pulses of drug after predetermined lag time period.

It is an another object of present invention to design a novel pulsatile drug delivery system having modifiable lag time for release of drug from dosage form before administration.

It is an another object of present invention to design a novel pulsatile drug delivery system, wherein a said novel pulsatile drug delivery system provide two or more different lag time of release of drug from dosage form.

It is an object of present invention to provide an afore said pulsatile drug delivery system, wherein lag time of drug release from dosage form is controlled by functional coating surrounding the core or composition of core or both.

It is an object of present invention to design a pulsatile drug delivery system that provide same or different lag time of release of one or more active substances from dosage form that are optimized with respect to therapeutic effect.

It is another object of present invention to provide a pulsatile drug delivery system, which is easy to manufacture.

### SUMMARY OF THE INVENTION

Method of treating disease condition with pulsatile drug delivery system comprising administration of intact dosage form after modifying lag time of release of drug; wherein said modified intact dosages form provide smaller lag time for release of drug from dosage form compared to administration of intact dosage form.

Method of treating disease condition with pulsatile drug delivery system comprising administration of intact dosage form after modifying lag time of drug release; wherein said modified intact dosages form provide smaller lag time for release of drug from dosage form compared to intact dosage form having predefined lag time for release of drug.

Method of treating disease condition with pulsatile drug delivery system having modifiable lag time for release of drug before administration is useful while patient miss administration of dosage form at particular time.

In preferred embodiment, a novel pulsatile drug delivery system having modifiable lag time for release of drug from dosage form before administration comprising:
1. a core comprising one or more active ingredient composition layer, wherein active ingredient composition layer comprises at least one active ingredients (A) and one or more pharmaceutically acceptable excipients (X); and
2. a functional coating surrounding a core.

In preferred embodiment, a novel pulsatile drug delivery system having modifiable lag time for release of drug from dosage form before administration comprising:
1. a core comprising one or more active ingredient composition layers and one or more time controlled composition layer; wherein
   (a) active ingredient composition layer comprises at least one active ingredients (A) and one or more pharmaceutically acceptable excipients (X),
   (b) time controlled composition layer comprises one or more pharmaceutically acceptable excipients (X); and
2. a functional coating surrounding a core.

In preferred embodiment, lag time of release of drug from active ingredients composition layer can be modified by making one or more aperture/cutting in composition layer or in coating before administration of intact dosage form. In preferred embodiment, when intact dosage form administer after making one or more aperture/cutting in composition layer or in functional coating provide smaller lag time for release of drug from active ingredient composition layer compared to intact dosage form. Intention of making an aperture in composition layer or in functional coating is to decrease lag time of drug release from active ingredients composition layer.

In preferred embodiment, a pulsatile drug delivery system may comprises one or more break lines or breaking regions at time controlled composition layer, wherein each break line or breaking region define the different lag time for release of drug from an active ingredient composition layer. In another embodiment, break line or breaking region closest to active ingredient composition layer provide smaller lag time for release of drug compared to break line or breaking region away from active ingredient composition layer.

In preferred embodiment, breaking region may also be defined by color of time controlled composition layer. In one embodiment, a pulsatile drug delivery system comprises two or more time controlled composition layer, wherein each time controlled composition layer comprises different coloring agent or coloring ingredients or combination of both to define breaking region. In one embodiment, each colored time controlled composition layer provide different lag time for release of drug or drugs from active ingredients composition layer.

In preferred embodiment, a novel pulsatile drug delivery system having modifiable lag time for release of drug from dosage form before administration comprising:
1. a core comprising one or more active ingredient composition layers, one or more time controlled composition layer and one or more swellable composition layer; wherein
   (a) active ingredient composition layer comprises at least one active ingredients (A) and one or more pharmaceutically acceptable excipients (X),
   (b) time controlled composition layer comprises one or more pharmaceutically acceptable excipients (X),
   (c) swellable composition layer comprises one or more pharmaceutically acceptable excipients (X); and
2. a functional coating surrounding a core.

In some embodiment, time controlled composition layer is being sandwich between active ingredients composition layer and swellable composition layer. In some embodiment, time controlled composition layer is being sandwich between two swellable composition layer.

In preferred embodiment, lag time of release of drug from intact dosage form can be modified by making one or more aperture/cutting in composition layer or in functional coating before administration of dosage form. In preferred embodiment, dosage form administered after making aperture/cutting in composition layer or in functional coating provides smaller lag time for release of drug from dosage form compared to intact dosage form. Intention of making aperture in composition layer or in functional coating is to decrease lag time of drug release from dosage form.

### BRIEF DESCRIPTION OF THE DRAWINGS

FIG. (1) is a diagrammatic representation of an embodiment of intact and modified dosage form of present invention which comprises: (I) a core comprising active ingredient composition layers, time controlled composition layer and swellable composition layer; and (II) a functional coating surrounding a core, wherein administration of intact dosage form after making aperture/cut in composition layer (modified intact dosage form) provide smaller lag time of drug release from dosage form compared to administration of intact dosage form.

FIG. (2) is a diagrammatic representation of an embodiment of intact and modified dosage form of present invention which comprises: (I) a core comprising active ingredient composition layers and time controlled composition layer; and (II) a functional coating surrounding a core, wherein administration of dosage form after making aperture/cut in composition layer (modified intact dosage form) provide smaller lag time of drug release from dosage form compared to administration of intact dosage form.

FIG. (3) is a diagrammatic representation of an embodiment of intact and modified dosage form of present invention which comprises: (I) a core comprising one active ingredient composition layers, and two time controlled composition layer, layer 1 and layer 2 respectively; and (II) a functional coating surrounding a core, wherein administration of dosage form after cutting from time controlled composition layer 1 or time controlled composition layer 2 (modified intact dosage form) provide smaller lag time of drug release from dosage form compared to administration of intact dosage form.

FIG. (4) is a diagrammatic representation of an embodiment of intact and modified dosage form of present invention which comprises: (I) a core comprising active ingredient composition layers and (II) a functional coating surrounding a core, wherein administration of dosage form after making aperture/cut in functional coating (modified intact dosage form) provide smaller lag time of drug release from dosage form compared to administration of intact dosage form.

### DETAILED DESCRIPTION OF THE INVENTION

Pulsatile drug delivery system is dosage form that release drug molecule in immediate release form or in controlled release form after a predetermined lag time period.

In the present invention, the term “lag time” is used to refer the time from contact of dosage form with an aqueous environment to time at which the active ingredient begins to get released from dosage form or from one or more active ingredient composition layers of dosage form.

In present invention, the term “water insoluble excipients or water insoluble polymer” is used to refer excipients or polymer which are insoluble in water at all pH or insoluble in water at certain pH such as e.g. Eudragit L100 55 is insoluble at pH below 5.5. So when the term “water insoluble excipients or water insoluble polymer” is specified, it is to be understood as it is insoluble in water at all pH or insoluble in water at certain pH.

In present invention, the term “water soluble excipients or water soluble polymer” is used to refer excipients or polymers which are soluble in water at all pH or soluble in water at certain pH such as e.g. Eudragit L100 55 is soluble at pH 5.5 or it above. So when the term “water soluble excipients or water soluble polymer” is specified, it is to be understood as it is soluble in water at all pH or soluble in water at certain pH.

In the present invention, “a time controlled composition layer” is used to refer composition layer that controlled the time for release of drug from active ingredient composition layer or from dosage form.

In the present invention, “active ingredient composition layer” is used to refer composition layer that comprising drug. Active ingredient composition layer release drug in immediate release form or in controlled release form or in combination of both. The term “controlled release” is used herein to refer a release which is later or slower than “immediate release”.

In the present invention, the term “composition layer” is used to refer active ingredient composition layer or time controlled composition layer or swellable composition layer or any two composition layer or all composition layer.

In the present invention, the term “break line or breaking region” is used to refer place or region from which time controlled composition layer is divisible. In general, breaking region is defined by two or more line.

Method of treating disease condition with pulsatile drug delivery system comprising administration of intact dosage form after modifying lag time of release of drug; wherein said modified intact dosages form provide smaller lag time for release of drug from dosage form compared to administration of intact dosage form.

Method of treating disease condition with pulsatile drug delivery system comprising administration of intact dosage form after modifying lag time of drug release; wherein said modified intact dosages form provide smaller lag time for release of drug from dosage form compared to intact dosage form having predefined lag time for release of drug.

Method of treating disease condition with pulsatile drug delivery system having modifiable lag time for release of drug before administration is useful while patient miss administration of dosage form at particular time.

For example, if patient need a drug at approximately 4 am on morning, then patient would administer dosage form having 8 hours lag time at around 8 pm on early night. But, if patient miss administration of dosage form at around 8 pm on early night and remember at around 11 pm, in such case dosage form is not useful because it deliver the drug at around 7 am on morning. At that time, modifiable lag time is useful to deliver drug before 7 am on morning.

In present invention, the term “intact dosage form” is used to refer dosage form having predefined lag time of drug release or dosage form without modifying lag time of drug release before administration or dosage form without any kind of manipulation before administration.

In present invention, the term “modified intact dosage form” is used to refer intact dosage form after modified lag time of drug release or intact dosage form after manipulation before administration. Modified intact dosage form provides smaller lag time of drug release compared to intact dosage form.

In preferred embodiment, a novel pulsatile drug delivery system having modifiable lag time for release of drug from dosage form before administration comprising:
1. a core comprising one or more active ingredient composition layer, wherein active ingredient composition layer comprises at least one active ingredients (A) and one or more pharmaceutically acceptable excipients (X); and
2. a functional coating surrounding a core.

In certain embodiment, a pulsatile drug delivery system is intact dosage form without any aperture/cutting in coating; wherein predefined lag time of release of drug from intact dosage form is particularly govern by coating surrounding the core or composition of core or combination of both.

In certain embodiment, when intact dosage form comes into contact with aqueous environment, aqueous fluid penetrate inside the dosage form through coating and hence active ingredient composition layer start to swell and break after predefined lag time resulting into release of drug. In certain embodiment, when intact dosage form comes into contact with aqueous environment, coating surrounding core is slowly start to dissolve or erode resulting into release of drug from active ingredients composition layer after predefined lag time.

In certain embodiment, lag time of release of drug from intact dosage form can be modifiable by making one or more aperture in coating before administration. In preferred embodiment, when intact dosage form administer after making one or more aperture/cut in coating provide smaller lag time for release of drug compared to intact dosage form having predefined lag time of release of drug. Intention of making aperture or cut in coating is to decrease lag time of drug release from dosage form while patient miss administration of dosage form at particular time period.

In preferred embodiment, a novel pulsatile drug delivery system having modifiable lag time for release of drug from dosage form before administration comprising:
1. a core comprising one or more active ingredient composition layers and one or more time controlled composition layer; wherein
   (a) active ingredient composition layer comprises at least one active ingredients (A) and one or more pharmaceutically acceptable excipients (X),
   (b) time controlled composition layer comprises one or more pharmaceutically acceptable excipients (X); and
2. a functional coating surrounding a core.

In preferred embodiment, a pulsatile drug delivery system is intact dosage form without any aperture/cutting in composition layer or in coating; wherein predefined lag time of release of drug from dosage form is particularly govern by functional coating surrounding the core or composition of core or combination of both.

In preferred embodiment, when intact dosage form comes into contact with aqueous environment, active ingredient composition layer start to swell and break after predefined lag time resulting into release of drug. In preferred embodiment, when intact dosage form comes into contact with aqueous environment, time controlled composition layer start to swell and break after predefined lag time resulting into release of drug from active ingredients composition layer.

In preferred embodiment, lag time of release of drug from active ingredients composition layer can be modified by making one or more aperture/cutting in composition layer or in coating before administration of intact dosage form. In preferred embodiment, when intact dosage form administer after making one or more aperture/cutting in composition layer or in functional coating provide smaller lag time for release of drug from active ingredient composition layer compared to intact dosage form. Intention of making an aperture in composition layer or in functional coating is to decrease lag time of drug release from active ingredients composition layer. In other word, drug delivery system provide two or more different lag time for release of same drug from single dosage form, i.e. actual predefined lag time of intact dosage form and modifiable lag time after manipulation with intact dosage form. Dosage form having modifiable lag time for release of drug is useful for patient when they miss administration of dosage form at particular time period. For example, if patient need a drug at approximately 4 am on morning, then patient would administer dosage form having 8 hours lag time at around 8 pm on early night. But, if patient miss administration of dosage form at around 8 pm on early night and remember at around 11 pm, in such case dosage form is not useful because it deliver the drug at around 7 am on morning. At that time, modifiable lag time is useful to deliver drug before 7 am on morning.

In preferred embodiment, a pulsatile drug delivery system may comprises one or more break lines or breaking regions at time controlled composition layer, wherein each break line or breaking region define the different lag time for release of drug from an active ingredient composition layer. In another embodiment, break line or breaking region closest to active ingredient composition layer provide smaller lag time for release of drug compared to break line or breaking region away from active ingredient composition layer. For example, intact dosage form with 8 hours of predefined lag time for release of drug comprises 3 breaking region at three different places on time controlled composition layer provides 2 h, 4 h and 6 h lag time for release of drug after breaking/cutting time controlled composition layer from the side of active ingredient composition layer, respectively.

In preferred embodiment, breaking region may also be defined by color of time controlled composition layer. In one embodiment, a pulsatile drug delivery system comprises two or more time controlled composition layer, wherein each time controlled composition layer comprises different coloring agent or coloring ingredients or combination of both to define breaking region. In one embodiment, each colored time controlled composition layer provide different lag time for release of drug or drugs from active ingredients composition layer.

In other words, pulsatile drug delivery system is flexible to deliver drug or drugs from dosage form after desire lag time.

In preferred embodiment, a novel pulsatile drug delivery system having modifiable lag time for release of drug from dosage form before administration comprising:
1. a core comprising one or more active ingredient composition layers, one or more time controlled composition layer and one or more swellable composition layer; wherein
   (a) active ingredient composition layer comprises at least one active ingredients (A) and one or more pharmaceutically acceptable excipients (X),
   (b) time controlled composition layer comprises one or more pharmaceutically acceptable excipients (X),
   (c) swellable composition layer comprises one or more pharmaceutically acceptable excipients (X); and
2. a functional coating surrounding a core.

In some embodiment, time controlled composition layer is being sandwich between active ingredients composition layer and swellable composition layer. In some embodiment, time controlled composition layer is being sandwich between two swellable composition layer.

In preferred embodiment, a pulsatile drug delivery system is intact dosage form without any aperture/cutting in composition layer or in coating; wherein predefined lag time of release of drug from intact dosage form is particularly govern by functional coating surrounding the core or composition of core or combination of both. In preferred embodiment, when intact dosage form comes into contact with aqueous environment, swellable composition layer start to swell and break after predefined lag time resulting into removal of swellable composition layer and coating that is in communication with swellable composition layer and hence at least one surface of time controlled composition layer is available for direct contact with aqueous environment through which aqueous fluid penetrate and reach in active ingredient composition layer resulting into release of drug from active ingredient composition layers after predefined period of time.

In preferred embodiment, lag time of release of drug from intact dosage form can be modified by making one or more aperture/cutting in composition layer or in functional coating before administration of dosage form. In preferred embodiment, dosage form administered after making aperture/cutting in composition layer or in functional coating provides smaller lag time for release of drug from dosage form compared to intact dosage form. Intention of making aperture in composition layer or in functional coating is to decrease lag time of drug release from dosage form. In one embodiment, when dosage form administered after making one or more aperture at swellable composition layer, aqueous fluid penetrate inside the swellable composition layer more rapidly thereby swellable composition layer start to swell and break more rapidly resulting into removal of swellable composition layer and coating that is in communication with swellable composition layer and hence at least one surface of time controlled composition layer is available rapidly or before predefined lag time for direct contact with aqueous environment resulting into decrease lag time for release of drug from active ingredient composition layers. In another embodiment, when dosage form administered after making one or more aperture at active ingredient composition layer, aqueous fluid penetrate inside the active ingredient composition layer more rapidly thereby active ingredient composition layer release drug immediately or before predefined lag time resulting into decrease lag time for release of drug from active ingredient composition layers.

In certain embodiment, a pulsatile drug delivery system comprises one or more active ingredient composition layers, wherein at least one active ingredient composition layer release the drug or drugs contained therein in immediate release manner or in controlled release manner or in combination of both. In certain embodiment, a pulsatile drug delivery system comprises two or more active ingredient composition layers, wherein composition in any one of active ingredient composition layer may be the same or different from composition in any of other active ingredient composition layer.

In certain embodiment, a pulsatile drug delivery system comprises two or more time controlled composition layers, wherein composition in any one of time controlled composition layer may be the same or different from composition in any of other time controlled composition layer. In certain embodiment, time controlled composition layer may optionally comprises drug or drugs.

In certain embodiment, a pulsatile drug delivery system comprises one or more swellable composition layer, wherein composition in any one of swellable composition layer may be the same or different from composition in any of other swellable composition layer. In certain embodiment, swellable composition layer may optionally comprises drug or drugs.

In preferred embodiment, a pulsatile drug delivery system comprises a functional coating surrounding a core in full or in part. In certain embodiment, a functional coating is either impermeable or permeable. In certain embodiment, functional coating comprises water insoluble ingredient or water soluble ingredient or mixture of water insoluble ingredient and water soluble ingredient. In one embodiment, a water soluble ingredient is selected from group consisting of pH dependent soluble excipients, pH independent soluble excipients, osmotic agent, pH modifying agent, natural or synthetic water soluble pharmaceutical polymer/excipient or any combination thereof. In certain embodiment, water soluble drug may also be used as water soluble ingredient.

In preferred embodiment, composition layer comprises at least one pharmaceutically acceptable excipients (X) selected from group consisting of release rate modifying agent, swellable excipients, gas generating agent, pH modifying agent, wicking agent, low density or high density excipients, pH dependent soluble excipients, pH independent soluble excipients, water insoluble excipients or any combination thereof.

In certain embodiment, a pulsatile drug delivery system may prepared by any kind of process such as compression of core and solvent coating, compression of core followed by compression coating, moulding, injection moulding, co-extrusion of the coating with core composition, extrusion or injection moulding and solvent coating and like.

In preferred embodiment, a pulsatile drug delivery system comprises a break lines in form of score line or printed line or other forms of indicia such as straight dotted lines, symbols or perforations may be placed at time controlled composition layer to define desired breaking region of drug delivery system. In certain embodiment, score line may be made by laser drilling.

In certain embodiment, a pulsatile drug delivery system comprises two or more active ingredient composition layer, wherein lag time to release drug or drugs from each active ingredient composition layer may be same or different. For example, pulsatile drug delivery system comprises two active ingredient composition layers, wherein lag time to release drug form both composition layer is approximately 4 hours or lag time to release drug from one active ingredient composition layer is approximately 4 hours and lag time to release drug from another active ingredient composition layer is approximately 8 hours. In other word, drug delivery system is flexible in controlling desire lag time for release of drug or drugs from each active ingredient composition layer.

In certain embodiment, lag time for release of drug or drugs from drug delivery system may controlled by functional coating surrounding the core, wherein said lag time is depend on rate of penetration of aqueous fluid inside the dosage form through functional coating or rate of erosion or solubilization of functional coating surrounding the core.

In certain embodiment, lag time for release of drug or drugs from dosage form controlled by composition of core, wherein said lag time is depend on type and concentration of excipients (X) used in drug delivery system.

### Components of Pulsatile Drug Delivery System—

### Active Substance:

The terms “drug”, “active substance”, “active ingredient”, “active agent”, “drug substance”, “pharmacologically active agent”, “therapeutically active substance”, “physiologically active agent” or “drug substance in physiologically active form” are used interchangeably herein to refer to a chemical compound that induces a desired pharmacological or physiological effect. The terms also encompass pharmaceutically acceptable derivatives of those active agents specifically mentioned herein, including, but not limited to, salts, solvates, polymorphs, hydrates, complexes with one or more molecules, prodrug, active metabolites, analogs, and the like. The letter “A” is used herein to denote “Active substance”. When the terms “drug”, “active substance”, “active ingredient”, “active agent”, “drug substance”, “pharmacologically active agent”, “therapeutically active substance”, “physiologically active agent” or “drug substance in physiologically active form” are used, or when a particular drug, for example metformine, is identified, it is to be understood as including the active agent as well as pharmaceutically acceptable salts, solvates, polymorphs, hydrates, complexes with one or more molecules, prodrugs, active metabolites, analogs and like.

In certain embodiments, drugs that may be used in the pharmaceutical composition of the present invention may be selected from the group consisting of, but are not limited to, of Alzheimer's disease, anesthetics, acromegaly agents, analgesics, centrally-acting analgesics, antiasthmatics, anabolic agents, appetite suppressants, anti-inflammatory, non-steroids anti-inflammatory, anticancer agents, anticoagulants and antithrombotic agents, anticonvulsants, antidiabetics, antiemetics, antipyretics, antiepileptics, antimigraine, antiglaucoma, antihistamines, anti-infective agents, antiparkinsons, antiplatelet agents, antirheumatic agents, antispasmodics and anticholinergic agents, antitussives, carbonic anhydrase inhibitors, cardiovascular agents, antiobesity, lipid modifying agents, cholinesterase inhibitors, treatment of CNS disorders, CNS stimulants, contraceptives, cystic fibrosis management, antipsychotics, dopamine receptor agonists, endometriosis management, anxiolytics, antidiarrheals, erectile dysfunction therapy, fertility agents, corticosteroid, gastrointestinal agents, Decongestant, cough suppressant, immunomodulators and immunosuppressives, memory enhancers, migraine preparations, muscle relaxants, anaesthetics, nucleoside analogues, opioids, osteoporosis management, adrenergics, parasympathomimetics, expectorants, antinauseants, prostaglandins, active substances against amoebiasis and other protozoal diseases; psychotherapeutic agents, antidepressants, sedatives, hypnotics and tranquilizers, drugs used for skin ailments, steroids and hormones and drugs used to treat narcolepsy and attention deficit hyperactivity disorder. In one embodiments, drugs that may be used in the pharmaceutical composition of the present invention may be selected from active substance associated with abuse syndromes include opioids, CNS depressants, CNS stimulants, cannabinoids, nicotine-like compounds, glutamate antagonists and N-methyl-D-aspartate (NMDA) antagonists.

The active substance can be in various forms, such as uncharged or charged molecules, molecular complexes, crystalline forms, amorphous form, polyamorphous form, polymorphous form, complexes, solvates, anhydrates, if relevant isomers, enantiomers, racemic mixtures and pharmacologically acceptable salts. Derivatives of active substances such as esters, ethers and amides which have solubility characteristics suitable for use herein can be used alone or mixed with other drugs. After release of the derivative from the drug delivery system it may be converted by enzymes, hydrolysed by body pH or other metabolic processes to the parent drug or to another biologically active form.

In one embodiment, a pharmaceutical composition of the invention may in addition be suitable for the delivery of peptides, polypeptides, hormones, proteins, antibodies and microorganisms, either living, attenuated or dead.

In preferred embodiment of present invention, the drug substance (A) may also include new chemical entity for which the amount of information is limited. In such cases, the dosage form regimen needs to evaluate based on preclinical and clinical trials.

### Swellable Excipients:

In certain embodiment, composition layer may comprise one or more swellable excipients/ingredients used in composition layer may be rapidly swellable, moderately swellable or slowly swellable. In certain embodiment, swellable excipient is selected based on requirement of dosage form property. The swellable excipients used in composition layer may be use in an amount ranging from about 0.5% to about 95% by weight of composition layer or dosage form. The swellable excipient that may be used in composition layer may be a rapidly swellable excipient may be selected from vinylpyrrolidone polymers such as crospovidone; cellulose and cellulose derivatives such as carboxyalkyl celluloses, crosslinked carboxyalkylcelluloses and their alkali salts; sodium starch glycolate, starch and starch derivatives, resins and like. The swellable excipient that may be used may be a rapidly swellable excipient and may be used in an amount ranging from about 0.5% to about 75% by weight of composition layer or dosage form, preferably about 1% to about 40% by weight of composition layer or dosage form. The swellable excipient that may be used in composition layer may be a moderately or slowly swellable excipient may be selected from cellulose derivative, starch derivatives, alginic acid and its copolymer, methacrylate and its copolymer, polyalkylene oxide and its copolymer, polyacrylic acid and its copolymer, gums of plant, animal, mineral or synthetic origin, carrageenan, natural or synthetic polymer or combination thereof. The swellable excipient that may be used may be a moderately or slowly swellable excipient and may be used in an amount ranging from about 4% to about 95% by weight of composition layer or dosage form, preferably about 10% to about 75% by weight of composition layer or dosage form.

### Gas Generating Agent:

The composition layers of drug delivery system may also comprise gas generating agent. Gas generating agents may helps in release of insoluble or poorly soluble drug and also in disintegration of composition layer. Gas generating agents that may be used in the present invention include carbonates such as calcium carbonate, bicarbonates such as sodium or potassium bicarbonate, sulfites such as sodium sulfite, sodium bisulfite, or sodium metabisulfite, and the like. These salts may be used alone or in combination with an acid source as a gas generating couple. The acid source may be an edible organic acid, a salt of an edible organic acid, acidic components such as acrylate polymers, or mixtures thereof. Examples of organic acids that may be used include citric acid, malic acid, succinic acid, tartaric acid, fumaric acid, maleic acid, ascorbic acid, glutamic acid, and their salts, and mixtures thereof.

### Wicking Agent:

The composition layers may further comprise a wicking agent in an amount ranging from about 0.5% to about 25% by weight of composition layer or dosage form. Examples of wicking agents that may be used include, but are not limited to, colloidal silicon dioxide, kaolin, titanium dioxide, fumed silicon dioxide, alumina, niacinamide, sodium lauryl sulfate, low molecular weight polyvinylpyrrolidone, m-pyrol, bentonite, magnesium aluminum silicate, polyester, polyethylene. Preferably, the wicking agents used in the pharmaceutical composition of the present invention include cellulose and cellulose derivatives, colloidal silicon dioxide, and mixtures thereof.

### Osmogent:

The composition layers may also comprise osmogents in an amount ranging from about 0.5% to about 50% by weight of composition layer or dosage form. Preferred osmogents include salts, acids, sugars and osmopolymer. Preferred salts include sodium chloride, potassium chloride, calcium chloride or magnesium chloride, lithium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate. Preferred acids include ascorbic acid, 2-benzene carboxylic acid, benzoic acid, fumaric acid, citric acid, maleic acid, serbacic acid, sorbic acid, edipic acid, edetic acid, glutamic acid, toluene sulfonic acid, water-soluble amino acids such as glycine, leucine, alanine, or methionine and tartaric acid; and like. Preferred sugars include dextrose, glucose, arabinose, ribose, arabinose, xylose, lyxose, xylol, allose, altrose, inosito, glucose, sorbitol, mannose, gulose, Glycerol, idose, galactose, talose, trehalose, mannitol, erythritol, ribitol, xylitol, maltitol, isomalt, lactitol, sucrose, raffinose, maltose, fructose, lactose, dextrin, dextran, amylase and xylan. These osmogents can be used alone or as a combination of two or more osmogents. Osmopolymers is selected from the group consisting of hydroxyethyl cellulose, poly(hydroxyalkyl methacrylate), poly (vinylpyrrolidone), poly(vinyl alcohol) having a low acetate content and lightly crosslinked with glyoxal, formaldehyde, glutaraldehyde and having a degree of polymerization from 2,000 to 30,000; poly(ethylene oxide), acidic carboxy polymers known as carboxypolymethylene or as carboxyvinyl polymers, a polymer consisting of acrylic acid lightly cross-linked with polyallylsucrose and Carbopol, acidic carboxy polymer having a molecular weight of 200,000 to 6,000,000, including sodium acidic carboxyvinyl hydrogel and potassium acidic carboxyvinyl hydrogel; polyacrylamide; and the like, and mixtures thereof.

### Release Rate Controlling Excipients:

In certain embodiment, composition layer may comprise one or more release rate modifying agents in an amount ranging from about 2% to about 95% by weight of composition layer or dosage form.

A suitable water soluble or water insoluble release rate modifying agent is selected from cellulose derivatives include, but are not limited to, methyl cellulose, hydroxypropyl methylcellulose, hydroxyethyl cellulose, hydroxypropyl cellulose, hydroxyethyl methylcellulose, cellulose acetate phthalate, microcrystalline cellulose, ethylhydroxyethylcellulose, ethylmethylcellulose, cellulose propionate, cellulose nitrate, cellulose acetate, hydroxymethylcellulose, hydroxymethylpropylcellulose, carboxymethylcellulose and sodium carboxymethylcellulose, ethyl cellulose; glycerol palmitostearate, wax like beeswax, glycowax, castor wax, carnauba wax, glycerol monostearate, hydrogenated vegetable oils, vegetable oils, stearyl alcohol, acetylated hydrogenated soybean oil glycerides, castor oil, glycerol behenic acid ester, glyceryl monooleate, glyceryl monostearate, propylene glycol monostearate, cetyl alcohol, natural and synthetic glycerides, fatty acids, fatty alcohol, lipid, steryl alcohol, sorbitan monolaurate, sorbitan monooleate, sorbitan monopalmitate, sorbitan monostearate, sorbitan sesquioleate, sorbitan trioleate, sorbitan tristearate, polyacrylamide derivatives, methacrylic acid derivatives, vinyl pyrrolidone polymers such as polyvinylpyrrolidone and copolymers of vinyl pyrrolidone and vinyl acetate, Polyalkylene oxide and copolymer thereof, alkylene oxide homopolymers; gums of plant, animal, mineral or synthetic origin, carageenan, polylactic acid or polyglycolic acid and copolymers thereof, methacrylates and its copolymer, Polyacrylic acid and copolymer thereof, a co-polymer of methacrylate-galactomannan etc., Polyvinyl alcohols, glycerinated gelatin, cocoa butter, macrogol esters, macrogol Stearate, phosphate esters, amides, phthalate esters, glyceryl cocoate oleyl alcohol, myristyl alcohol, sucrose octaacetate, diacetylated monoglycerides, diethylene glycol monostearate, ethylene, polyoxyethylene 50 stearate, macrogol ethers, cetomacrogol 1000, lauromacrogols, poloxamers, and mixtures thereof.

### pH Modifying Agent:

In certain embodiment, a pulsatile drug delivery system comprises pH modifying agent which include pH modifying basic agent or pH modifying acidic agent or both. In some embodiment, antacid may be used as pH modifying basic agent.

pH modifying basic agent include, but are not to be limited, magnesium oxide, meglumine, sodium oxide, sodium hydroxide, sodium bicarbonate, bismuth carbonate, bismuth subcarbonate, bismuth subgallate, bismuth subnitrate, potassium citrate, sodium citrate, sodium carbonate, potassium bicarbonate, potassium carbonate, calcium carbonate, calcium phosphate, dibasic calcium phosphate, dihydroxyaluminum aminoacetate, dihydroxyaluminum sodium carbonate, glycine, magnesium glycinate, magnesium hydroxide, magnesium carbonate, sodium borate, aluminum oxide, aluminum hydroxide, ammonium Carbonate, monoethanolamine, diethanolamine, triethanolamine, Potassium Hydroxide, Sodium Phosphate Dibasic, Trolamine, sodium potassium tartrate, tribasic sodium phosphate, tricalcium phosphate and like.

pH modifying acidic agent include, but are not to be limited, ascorbic acid, benzoic acid, boric acid, citric acid, EDTA and derivative thereof like disodium edetate, trisodium edetate, tetrasodium edetate, disodium calcium edetate and like, edetic acid, erythrobic acid, fumaric acid, lactic acid, lauric acid, linoleic acid, malic acid, alginic acid, myristic acid, oleic acid, palmitic acid, sorbic acid, succinic acid, tartaric acid and like.

### pH Dependent Soluble Ingredient:

In certain embodiment, a pulsatile drug delivery system comprises pH dependent soluble ingredient.

In some embodiment, pH dependent soluble ingredient which are soluble at pH of stomach fluid or pH value less than about 5.5 include, but are not to be limited, calcium carbonate, chitin, chitosan, di and tribasic calcium phosphate, magnesium hydroxide, polymethacrylate or methacrylic acid and its derivative such as dimethylaminoethyl methacrylate, butyl methacrylate, and methyl methacrylate, for example Eudragit EPO, Eudragit E100, Eudragit 12.5 and like.

In some embodiment, pH dependent soluble ingredient which are soluble at pH of intestinal fluid or pH value greater than about 5.5 include, but are not limited, polymethacrylate such as eudragit S 100, eudragit L 100, eudragit L 100-55 or mixture thereof, cellulose esters such as cellulose acetate phthalate, cellulose acetate trimellitate, hydroxypropylmethyl cellulose acetate succinate, HPMC phthalate, polyvinyl derivative such as polyvinyl acetate phthalate, shellac and like.

### pH Independent Soluble Excipients:

According to present invention, pH independent soluble excipients include natural, synthetic or semi synthetic excipients. A said pH independent soluble are selected from group consisting of polymer, sugar, salts, salts of organic acid, acid and polysaccharide. pH independent soluble polymer include, but are not to be limited, cellulose derivatives include, but are not limited to be, methyl cellulose, hydroxypropyl methylcellulose, hydroxyethyl cellulose, hydroxypropyl cellulose, hydroxyethyl methylcellulose, ethylhydroxyethylcellulose, ethylmethylcellulose, hydroxymethylcellulose, hydroxymethylpropylcellulose, sodium carboxymethylcellulose, polyacrylamide derivatives, methacrylic acid derivatives; vinyl pyrrolidone polymers such as polyvinylpyrrolidone; Starch derivative, Polyalkylene oxide and copolymer thereof, alkylene oxide homopolymers; gums of plant, animal, mineral or synthetic origin, methacrylates, Polyacrylic acid and copolymer thereof, Polyvinyl alcohols, polyethylene glycol, poloxamer; and mixtures thereof. Preferred sugars include dextrose, glucose, arabinose, ribose, arabinose, xylose, lyxose, xylol, allose, altrose, inositol, glucose, sorbitol, mannose, gulose, Glycerol, idose, galactose, talose, trehalose, mannitol, erythritol, ribitol, xylitol, maltitol, isomalt, lactitol, sucrose, raffinose, maltose, fructose, lactose, dextrin, dextran, amylase and xylan. Preferred salts include sodium chloride, potassium chloride, calcium chloride or magnesium chloride, lithium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate. Preferred acids include ascorbic acid, 2-benzene carboxylic acid, benzoic acid, fumaric acid, citric acid, maleic acid, serbacic acid, sorbic acid, edipic acid, edetic acid, glutamic acid, toluene sulfonic acid, water-soluble amino acids such as glycine, leucine, alanine, or methionine and tartaric acid; and like. Polysaccharides are polymeric carbohydrate molecules composed of long chains of monosaccharide units bound together by glycosidic linkages and on hydrolysis give the constituent monosaccharides or oligosaccharides. They range in structure from linear to highly branched. Examples include storage polysaccharides such as starch and glycogen, and structural polysaccharides such as cellulose.

### Water Insoluble Excipients:

According to present invention, water insoluble excipients include natural, synthetic or semi synthetic water insoluble material. Natural, synthetic or semi synthetic water insoluble material include, but are not to be limited, cellulose derivatives include cellulose acetate phthalate, microcrystalline cellulose, cellulose acetate, HPMC phthalate, microcrystalline cellulose, ethyl cellulose; glycerol palmitostearate, Wax include microcrystalline wax, beeswax, glycowax, castor wax, carnauba wax; glycerol monostearate, hydrogenated vegetable oils, vegetable oils, stearyl alcohol, acetylated hydrogenated soybean oil glycerides, castor oil, glycerol behenic acid ester, glyceryl monooleate, glyceryl monostearate, propylene glycol monostearate, cetyl alcohol, natural and synthetic glycerides, fatty acids, fatty alcohol, lipid, steryl alcohol, sorbitan monolaurate, sorbitan monooleate, sorbitan monopalmitate, sorbitan monostearate, sorbitan sesquioleate, sorbitan trioleate, sorbitan tristearate, polyacrylamide derivatives, methacrylic acid derivatives such polymethaacrylate and its copolymer; Polyvinyl Acetate, copolymers of vinyl pyrrolidone and vinyl acetate; vinyl acetate and copolymer thereof, ethyl vinyl acetate, modified starch like pregelatinised starch, polylactic acid or polyglycolic acid and copolymers thereof, methacrylates, Polyacrylic acid and copolymer thereof, a co-polymer of methacrylate-galactomannan etc., cocoa butter, macrogol Stearate, diethylene glycol monostearate, polyoxyethylene 50 stearate, dibasic calcium phosphate and mixtures thereof.

### Low Density or High Density Excipient

Suitable low density materials include, but are not to be limited, polyvinyl alcohol-polyethylene glycol graft copolymers, acrylic acid polymers, Wax, methacrylic acid copolymers, polyvinyl alcohol, polyvinyl acetate, polysaccharides, cellulose based polymers or combinations thereof. High density materials include, but are not to be limited, barium sulphate, zinc oxide, iron powder, titanium dioxide etc.

### EXAMPLES

Certain aspects of the present invention may be better understood as illustrated by the following examples, which are meant by way of illustration and not limitation.

### Example 1

| | | |
|---|---|---|
| Sr. No | Ingredients Name | Qty/tab (% w/w) |

Active ingredient composition layer

| | | |
|---|---|---|
| 1 | Sertraline HCl | 18.18 |
| 2 | Sodium Starch Glycolate | 4.55 |
| 3 | Mannitol | 7.82 |
| 4 | Microcrystalline Cellulose | 14.45 |
| 5 | Magnesium Stearate | 0.45 |

Time controlled composition layer

| | | |
|---|---|---|
| 6 | Ethyl Cellulose | 13.64 |
| 7 | Mannitol | 9.09 |

Swellable composition layer

| | | |
|---|---|---|
| 8 | Crospovidone | 4.55 |
| 9 | Microcrystalline Cellulose | 15.90 |
| 10 | Sodium Lauryl Sulphate | 1.82 |
| 11 | Magnesium Stearate | 0.45 |

Functional coating

| | | |
|---|---|---|
| 12 | Ethylcellulose aqueous dispersion | 6.60 |
| 13 | Dibutyl Sebacate | 1.40 |
| 14 | Mannitol | 0.55 |
| 15 | Povidone K 30 | 0.55 |

### Procedure

### Trilayer Tablet Preparation:

1. Ingredients of each composition layer were weighed and sifted through ASTM 40# sieve.
2. Single layer core tablets of each composition layer were compressed at low hardness.
3. Trilayer tablet was prepared by adding single layer core tablets of each composition layer in compression die in following sequence—
   a) Single layer core tablets of active ingredient composition layer
   b) Single layer core tablets of time controlled composition layer
   c) Single layer core tablets of swellable composition layer
4. Final compression of step (4) was performed at target hardness.

### Functional Coating:

5. Coating solution was prepared as follow:
   i. Dibutyl sebacate was added in ethyl cellulose aqueous dispersion and stirred for 30 minutes.
   ii. Mannitol and Povidone K 30 were added in water and stirred for 30 minutes.
   iii. Step (b) solution was added in step (a) dispersion and it was stirred for another 30 minutes.
6. Step (4) trilayer core tablets were coated with step (5) functional coating solution.

### Example 2

| | | |
|---|---|---|
| Sr. No | Ingredients Name | Qty/tab (% w/w) |

Active ingredient composition layer

| | | |
|---|---|---|
| 1 | Sertraline HCl | 23.52 |
| 2 | Crospovidone | 6.89 |
| 3 | Mannitol | 9.12 |
| 4 | Microcrystalline Cellulose | 18.70 |
| 5 | Magnesium Stearate | 0.58 |

Time controlled composition layer

| | | |
|---|---|---|
| 6 | Ethyl Cellulose | 11.65 |
| 7 | Mannitol | 17.76 |

Functional coating

| | | |
|---|---|---|
| 8 | Ethylcellulose aqueous dispersion | 8.54 |
| 9 | Dibutyl Sebacate | 1.81 |
| 10 | Mannitol | 0.71 |
| 11 | Povidone K 30 | 0.71 |

### Procedure

### Bilayer Tablet Preparation:

1. Ingredients of each composition layer were weighed, sifted through ASTM 40# sieve and mixed in polybag.
2. Bilayer tablet was prepared by adding blend of each composition layer in compression die in following sequence—
   i. blend of active ingredient composition layer
   ii. blend of time controlled composition layer
3. Final compression of step (2) was performed at target hardness.
4. Functional coating:
5. Coating solution was prepared as follow:
   i. Dibutyl sebacate was added in ethyl cellulose aqueous dispersion and stirred for 30 minutes.
   ii. Mannitol and Povidone K 30 were added in water and stirred for 30 minutes.
   iii. Step (b) solution was added in step (a) dispersion and it was stirred for another 30 minutes.
6. Step (3) bilayer core tablets were coated with step (4) functional coating solution.

1-12. (canceled)

13. A pulsatile drug delivery system having modifiable lag time of release of drug from dosage form before administration comprising:
(a) a core comprising at least one active ingredient and one or more pharmaceutical acceptable excipients; and
(b) a functional coating surrounding a core;
wherein said pulsatile drug delivery system is useful in treating disease condition when patient miss administration of intact dosage form at particular time period.

14. (canceled)

15. (canceled)

16. (canceled)

17. A pulsatile drug delivery system having modifiable lag time of release of drug from dosage form before administration of claim 13, wherein lag time of release of drug from dosage form is governed by at least one of composition of core and functional coating.

18. A pulsatile drug delivery system having modifiable lag time of release of drug from dosage form before administration of claim 13, wherein lag time of drug release from intact dosage form is modifiable before administration by making aperture in composition layer or in functional coating or by cutting composition layer or functional coating.

19. A pulsatile drug delivery system having modifiable lag time of release of drug from dosage form before administration of claim 13 provide two or more different lag time for release of drug from dosage form.

20. A pulsatile drug delivery system having modifiable lag time of release of drug from dosage form before administration of claim 13, wherein a said drug delivery system comprising at least one drug substance and one or more pharmaceutically acceptable excipients selected from group consisting of release rate modifying agent, swellable excipients, gas generating agent, pH modifying agent, wicking agent, low density or high density excipients, pH dependent soluble excipients, pH independent soluble excipients, and water insoluble excipients.

21. A pulsatile drug delivery system having modifiable lag time of release of drug from dosage form before administration of claim 13, wherein core is single layer.

22. A pulsatile drug delivery system having modifiable lag time of release of drug from dosage form before administration of claim 13, wherein core is multi layers.

23. A pulsatile drug delivery system having modifiable lag time of release of drug from dosage form before administration of claim 22, wherein one layer is active ingredient composition layer and another layer is at least one of time controlled composition layer and swellable composition layer.

24. A pulsatile drug delivery system having modifiable lag time of release of drug from dosage form before administration provide short lag time for drug release from modified intact dosage form after administration compared to lag time of drug release from intact dosage form after administration; wherein said modified intact dosage form is useful in treating disease condition when patient miss administration of intact dosage form at particular time period.
